(19) United States
(12) Patent Application Publication
(10) Pub. No.: US 2003/0092638 A1
(43) Pub. Date: May 15, 2003

(54) Protopanaxadiol and protopanaxatriol and their use as anti-cancer agents

(76) Inventors: Dong Huang, Surrey (CA); Dong Feng Qi, Shenyang City (CN)

Correspondence Address: Gerald O.S. Oyen, Oyen Wiggs Green & Mutala, # 480 - 601 West Cordova Street, Vancouver, BC, V6B 1G1, CA

(21) Appl. No.: 09/957,082

(22) Filed: Sep. 21, 2001

(51) Int. Cl. A61K 31/704; A61K 31/337

(52) U.S. Cl. 514/26; 514/449

(57) ABSTRACT

The invention provides for the use of sapogenin protopanaxadiol and/or sapogenin protopanaxatriol in synergistic combination with another cancer chemotherapeutic in pharmaceutical compositions used in methods of inhibiting the multiplication of cancer cells, and methods of treating cancer in patients, comprising administering to such patients therapeutically and synergistically effective amounts of sapogenin protopanaxadiol and/or sapogenin protopanaxatriol in combination with a chemotherapeutic selected from the group consisting of paclitaxel and mitoxantrone. The cancer cells to be treated may be multi-drug resistant. The cancer cells may, for example, be prostate cancer cells or breast cancer cells.

### FIELD OF THE INVENTION

This invention relates to a novel combination of sapogenin protopanaxadiol and/or protopanaxatriol and other anti-cancer agents, and the use of this combination in cancer treatment applications.

### BACKGROUND OF INVENTION

Since the beginning of the last decade, anti-cancer research has been increasingly directed to the discovery of novel anti-cancer agents obtained from natural sources, as well as identifying and preparing synthetic compounds found in natural sources.

Ginseng saponins (dammarane saponins, also called “ginsenosides”, which are effective ingredients that organically exist in panax ginseng, panax quinguefol, panax notoginseng and other species in the ginseng family) and sapogenins (those that do not naturally exist in the ginseng plant or other species in the ginseng family and can be derived only through chemical structure modification by cleavage and/or semi-synthesis of dammarane saponins), as natural-source root compounds, have been broadly researched for their anti-cancer characteristics. Some of them have been reported to have anti-cancer effects, of which, for example, ginsenoside Rh2 [3-O-β-D-glucopyranosyl-20(s)-protopanaxadiol] has been reported for its anti-cancer activities [1], including induction of differentiation and apoptosis in cancer cells [5^{˜}11], inhibition of the growth of human ovarian cancer in nude mice after oral administration [9], and the ability to inhibit the multiplication of multi-drug resistance (MDR) cancer cells while used with other chemotherapy drugs in vitro [12].

Ginsenoside Rg3 [3-O-[β-D-glucopyranosyl(1→2)-β-D-glucopyranosyl]-20(s)-protopanaxadiol] has been reported to inhibit the invasion by various cancer cells [13] and suppress the proliferation of human prostate cancer cells [14] in vitro, and to inhibit lung metastasis in mice [15] and peritoneal metastasis in rats [16].

A metabolite of ginseng saponin produced by human intestinal bacteria, Mc [20-O-[α-L-arabinofuranosyl (1→6)-β-D-glucopyranosyl]-20(s)-protopanaxadiol], has been reported to inhibit the vascularization of tumors and extravasation of cancer cells [17].

While conventional chemotherapy agents directly attack the cancer cells and exhibit severe adverse side effects, some ginseng saponins and sapogenins, as well as their intestinal bacteria metabolites, have been reported to have inhibitory effects on cancers by induction of cancer-cell apoptosis and/or by suppression of vascularization of cancers with few adverse side effects.

In the case of treatment of cancers with ginseng saponins, it has been reported that saponins which are metabolized to sapogenins by intestinal bacteria have anti-cancer effects.

Mitoxantrone is an antineoplastic agent which may be prepared as a synthetic antracenedione derivative of the anthraquinone dye ametantrone. Mitoxantrone has the following formula:

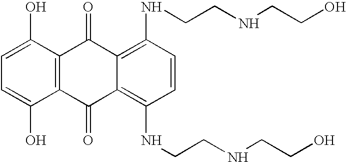

Mitoxantrone has reportedly been used in the treatment of a variety of malignant diseases, including acute non-lymphocytic leukemia, advanced breast cancer and prostate cancer (see Wiseman and Spencer, Drugs & Aging, 1997 June 10(6):473). Mitoxantrone is available commercially, and its preparation is, for example, described in U.S. Pat. No. 4,197,249.

Paclitaxel is a derivatized diterpenoid which may be obtained from the bark of the Pacific Yew and other natural sources (Taxus brevifolia, see Wani et al., J. Am. Chem. Soc. 93:2325, 1971; and Stierle et al., Science 60:214-216, 1993). Therapeutically, particularly in cancer therapy, paclitaxel is thought to act to stabilize microtubular structures by binding tubulin. As used herein, the word “paclitaxel” may include analogues, derivatives and conjugates of the naturally-occurring molecule, such as TAXOL™, TAXOTERE™, 10-desacetyl analogues of paclitaxel, 3′N-desbenzoyl-3′N-t-butoxy carbonyl analogues of paclitaxel, paclitaxel-PEG, paclitaxel-dextran, or paclitaxel-xylos. Paclitaxel may be prepared utilizing a variety of techniques (see WO 94/07882, WO 94/07881, WO 94/07880, WO 94/07876, WO 93/23555, WO 93/10076, U.S. Pat. Nos. 5,294,637, 5,283,253, 5,279,949, 5,274,137, 5,202,448, 5,200,534, 5,229,529, and EP 590267), or obtained from a variety of commercial sources, including for example, Sigma Chemical Co., St. Louis, Mo.

Cisplatin is an inorganic compound possessing a platinum element.

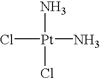

Cisplatin (cis-diaininedichloroplatinum (II)) has been used as a chemotherapeutic agent for many years since discovery of its anti-tumor activity (Rosenberg et al., Nature, 205:698, 1965; Nature 222:385, 1972; U.S. Pat. No. 4,177,263). The mechanism of action of cisplatin in cancer therapy is believed to be through its ability to bind to DNA to interfere with repair mechanisms, causing cell death. Cisplatin has been reported to be effective in the treatment of a variety of cancers, most significantly in the treatment of ovarian and testicular cancer. Cisplatin is available commercially, for example, from Bristol-Myers Squibb under the commercial name PLATINOL™.

Cancerous tumors that have responded well initially to a particular drug or drugs, may later develop a tolerance to the drug(s) and cease responding, a phenomenon known as multi-drug resistance. Multi-drug resistance is generally characterized by cross-resistance of a disease such as cancer to more than one functionally or structurally unrelated drugs. Multi-drug resistance may be caused by a number of mechanisms. For example, multi-drug resistance may be mediated by a protein that is variously called multi-drug-resistance 1 protein (MDR1), pleiotropic-glycoprotein (P-glycoprotein), Pgp or P170, referred to herein as “P-glycoprotein”. P-glycoprotein is thought to be endogenous in cell membranes in certain drug resistant cells, multi-drug resistant tumor cells, gastrointestinal tract cells, and the endothelial cells that form the blood brain barrier, where P-glycoprotein is thought to act as an efflux pump for the cell. Certain substances, including treatment drugs for various diseases, may be pumped out of the cell by the P-glycoprotein prior to having a therapeutic effect on the cell. There is accordingly a need for therapeutic approaches that may be used to counteract drug resistance, particularly multi-drug resistance mediated by P-glycoprotein in cancer.

### SUMMARY OF THE INVENTION

In one aspect, the invention provides methods inhibiting the multiplication of cancer cells, and methods of treating cancer in patients in need of such treatment, comprising administering to such patients therapeutically and synergistically effective amounts of sapogenin protopanaxadiol and/or sapogenin protopanaxatriol in combination with a chemotherapeutic selected from the group consisting of paclitaxel and mitoxantrone. The cancer cells to be treated may be multi-drug resistant. The cancer cells may for example be prostate cancer cells or breast cancer cells.

The chemical structures of protopanaxadiol and protopanaxatriol are as follows:

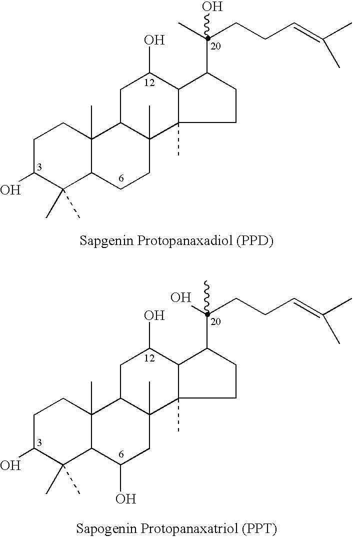

In alternative aspects, the invention provides for the use of sapogenin protopanaxadiol and/or sapogenin protopanaxatriol in combination with a chemotherapeutic selected from the group consisting of paclitaxel and mitoxantrone to synergistically inhibit the multiplication of cancer cells, or to formulate a medicament for inhibiting the multiplication of cancer cells synergistically. The cancer cells to be treated may be multi-drug resistant. The cancer cells may, for example, be prostate cancer cells or breast cancer cells.

In alternative aspects, the invention provides for the use of sapogenin protopanaxadiol and/or sapogenin protopanaxatriol to render non-P-glycoprotein multi-drug resistant cancer cells (cancer cells that do not express P-glycoprotein) sensitive to a chemotherapeutic. In such embodiments, a chemotherapeutic may, for example, be used with sapogenin protopanaxadiol and sapogenin protopanaxatriol to treat a patient at a concentration or dosage at which the chemotherapeutic alone would otherwise not be effective in said non-P-glycoprotein multi-drug resistant cancer cells. The chemotherapeutic may, for example, be paclitaxel, mitoxantrone, or cisplatin. The cancer cells may, for example, be prostate cancer cells or breast cancer cells.

In alternative aspects, the invention provides a pharmaceutical composition for the treatment of cancer, in patients in need of such treatment, comprising a pharmaceutically acceptable carrier and therapeutically and synergistically effective amounts of sapogenin protopanaxadiol and/or sapogenin protopanaxatriol and a chemotherapeutic selected from the group consisting of paclitaxel and mitoxantrone.

The invention is directed to a method of inhibiting the multiplication of cancer cells by treating the cells with a therapeutically and synergistically effective combination of paclitaxel, and sapogenin protopanaxadiol and/or sapogenin protopanaxatriol.

The invention also pertains to a method of inhibiting the multiplication of cancer cells by treating the cells with a therapeutically and synergistically effective combination of mitoxantrone, and sapogenin protopanaxadiol and/or sapogenin protopanaxatriol.

The cancer cells can be multi-drug resistant and need not express P-glycoprotein. The cancer cells can be selected from the group consisting of prostate cancer cells and breast cancer cells.

The invention also includes a method of treating a patient having a cancer with a therapeutically and synergistically effective combination of paclitaxel and sapogenin protopanaxadiol and/or sapogenin protopanaxatriol, and a method of treating a patient having a cancer with a therapeutically and synergistically effective combination of mitoxantrone, sapogenin protopanaxadiol and/or sapogenin protopanaxatriol.

The invention includes the use of a therapeutically effective amount of sapogenin 20(R) protopanaxadiol and sapogenin 20(S) protopanaxatriol in combination with paclitaxel or in combination with mitoxantrone to synergistically inhibit the multiplication of cancer cells.

The invention also includes the use of sapogenin protopanaxadiol and/or sapogenin protopanaxatriol to formulate a medicament for inhibiting the multiplication of cancer cells synergistically with paclitaxel or mitoxantrone.

The invention is also directed to a method of inhibiting the multiplication of multi-drug resistant cancer cells, wherein the cells do not express P-glycoprotein, comprising treating the cells with a therapeutically and synergistically effective combination of sapogenin protopanaxadiol and/or sapogenin protopanaxatriol and a chemotherapeutic. The chemotherapeutic can be paclitaxel, mitoxantrone or cisplatin. The multi-drug resistant cancer cells can be selected from the group consisting of prostate cancer cells and breast cancer cells.

The invention also pertains to the use of a therapeutically effective amount of sapogenin protopanaxadiol and/or sapogenin protopanaxatriol in combination with a chemotherapeutic to inhibit the multiplication of multi-drug resistant cancer cells, wherein the cells do not express P-glycoprotein, and the chemotherapeutic alone is not therapeutically effective to inhibit the multiplication of the cancer cells. The chemotherapeutic can be paclitaxel or mitoxantrone or cisplatin. The multi-drug resistant cancer cells can be selected from the group consisting of prostate cancer cells and breast cancer cells.

The invention is also directed to a pharmaceutical composition comprising a pharmaceutically acceptable carrier, and therapeutically and synergistically effective amounts of sapogenin protopanaxadiol and/or sapogenin protopanaxatriol, and a chemotherapeutic selected from the group consisting of paclitaxel and mitoxantrone.

The form of the composition can be selected from the group consisting of an orally administrable form, an injectable form, and an externally applicable form. The composition can be the orally administrable form and selected from the group consisting of a tablet, a powder, a suspension, an emulsion, a capsule, a granule, a troche, a pill, a liquid, a spirit, a syrup or a limonade, or it can be the injectable form and selected from the group consisting of a liquid, a suspension and a solution. The composition can also be the externally applicable form selected from the group consisting of an ointment, a liquid, a powder, a plaster, a suppository, an aerosol, a liniment, a lotion, an enema and an emulsion.

### DRAWINGS

In drawings which illustrate specific embodiments of the invention, but which should not be construed as restricting the spirit or scope of the invention in any way:

FIG. 1 is a graph illustrating a comparison of cytotoxicity of sapogenin protopanaxadiol (PPD), sapogenin protopanaxatriol (PPT) and Rh2, showing cellular viability of B16 melanoma tumor cells on the vertical axis and the chemotherapeutic concentration on the horizontal axis.

FIGS. 2a and 2b are two graphs, A and B, showing the use of sapogenin protopanaxadiol (PPD) and sapogenin protopanaxatriol (PPT) in combination with paclitaxel on breast cancer cells MCF7/MCF7r and showing cellular viability on the vertical axis and paclitaxel concentration on the horizontal axis, with four lines on each graph representing paclitexal only, and paclitexal with Rh2, PPD and PPT.

### DETAILED DESCRIPTION OF THE INVENTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

Sapogenins do not exist in ginseng. Saponins which exist in ginseng must be converted by chemical reaction into sapogenins. Cancers susceptible to treatment with sapogenin protopanaxadiol and sapogenin protopanaxatriol in combination with a chemotherapeutic 25 in accordance with various aspects of the invention may include both primary and metastatic tumors and hyperplasias, including carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract, (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas). In some aspects of the invention, sapogenin protopanaxadiol and sapogenin protopanaxatriol in combination with a chemotbrapeutic may also be useful in treating hematopoietic cancers such as leukemias (i.e. chloromas, plasmacytomas and the plaques and tumors of mycosis fungoides and cutaneous T-cell lymphomal leukemia) and lymphomas (both Hodgkin's and non-Hodgkin's lymphomas). In addition, sapogenin protopanaxadiol and sapogenin protopanaxatriol in combination with a chemothrapeutic may be useful in the prophylactic prevention of metastases from the tumors described above.

Sapogenin protopanaxadiol and/or sapogenin protopanaxatriol and the chemotherapeutic may be administered in combination separately or as one single combined pharmaceutical composition. The amount of each component administered may be determined by an attending clinician, taking into consideration a variety of factors such as the etiology and severity of the disease, the patient's condition and age and the potency of each component. The components may be administered in accordance with the standard methodologies as for example disclosed in the Physician's Desk Reference (PDR) published by Medical Economics Co. Inc. of Oradell, N.J.

A therapeutically and synergistically effective combination of sapogenin protopanaxadiol and/or sapogenin protopanaxatriol and a chemotherapeutic is characterized by the fact that the chemotheraputic is administered at a chemotherapeutic dosage and sapogenin protopanaxadiol and/or sapogenin protopanaxatriol is administered at a therapeutic dosage, and the therapeutic effect thereby achieved, such as inhibition of cellular multiplication, is greater than the sum of the therapeutic effect that would be achieved with the chemotherapeutic alone at the chemotherapeutic dosage plus the therapeutic effect that would be achieved with sapogenin protopanaxadiol and/or sapogenin protopanaxatriol alone at the therapeutic dosage. For example, a therapeutically and synergistically effective combination of sapogenin protopanaxadiol and/or sapogenin protopanaxatriol and paclitexal is a combination wherein the paclitexal is administered at a paclitexal dosage and sapogenin protopanaxadiol and/or sapogenin protopanaxatriol is administered at a therapeutic dosage, and the inhibition of cellular multiplication thereby achieved is greater than the sum of the inhibition that would be achieved with paclitexal alone at the paclitexal dosage plus the inhibition that would be achieved with sapogenin protopanaxadiol and/or sapogenin protopanaxatriol alone at the therapeutic dosage. Similarly, a therapeutically and synergistically effective combination of sapogenin protopanaxadiol and/or sapogenin protopanaxatriol and mitoxantrone is a combination wherein the mitoxantrone is administered at a mitoxantrone dosage and sapogenin protopanaxadiol and/or sapogenin protopanaxatriol is administered at a therapeutic dosage, and the inhibition of cancer cell multiplication thereby achieved is greater than the sum of the inhibition that would be achieved with mitoxantrone alone at the mitoxantrone dosage plus the inhibition that would be achieved with sapogenin protopanaxadiol and/or sapogenin protopanaxatriol alone at the therapeutic dosage.

One or more pharmaceutically acceptable carriers or exipients may be used to formulate pharmaceutical compositions of the invention, including solvents, dispersion media, coatings, antibacterial and antifungal agents, isotonic and absorption delaying agents, and the like that are physiologically compatible. In alternative embodiments, the carrier may be suitable for parenteral, intravenous, intraperitoneal, intramuscular, sublingual or oral administration. Pharmaceutically acceptable carriers may include sterile aqueous solutions or dispersions and sterile powders for the extemporaneous preparation of sterile injectable solutions or dispersion. Except insofar as any conventional media or agent is incompatible with the active compound, use thereof in the pharmaceutical compositions of the invention is contemplated. Supplementary active compounds can also be incorporated into the pharmaceutical compositions.

Pharmaceutical compositions typically must be sterile and stable under the conditions of manufacture and storage. The composition may be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the pharmaceutical compositions may be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglyeolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating an active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. Pharmaceutical compositions may be formulated with one or more compounds that enhance the solubility of the active compounds.

Procedures for the isolation and purification of sapogenin protopanaxadiol and/or sapogenin protopanaxatriol may for example include aqueous or organic extraction, column chromatography, thin-layer chromatography, and high performance chromatography. Techniques for the extraction and purification of plant extracts may be adapted for the preparation of sapogenin protopanaxadiol and/or sapogenin protopanaxatriol from the root of Panax ginseng, such as techniques disclosed in the following documents (which are incorporated herein by reference): U.S. Pat. No. 6,156,291 issued to Pang, et al. on Dec. 5, 2000; U.S. Pat. No. 6,083,932 issued to Pang, et al. on Jul. 4, 2000; U.S. Pat. No. 4,157,894 issued to Bombardelli on Jun. 12, 1979; U.S. Pat. No. 5,137,878 issued to Pang, et al. on Aug. 11, 1992; U.S. Pat. No. 5,230,889 issued to Inoue on Jul. 27, 1993; U.S. Pat. No. 5,589,182 issued to Tashiro, et al. on Dec. 31, 1996.

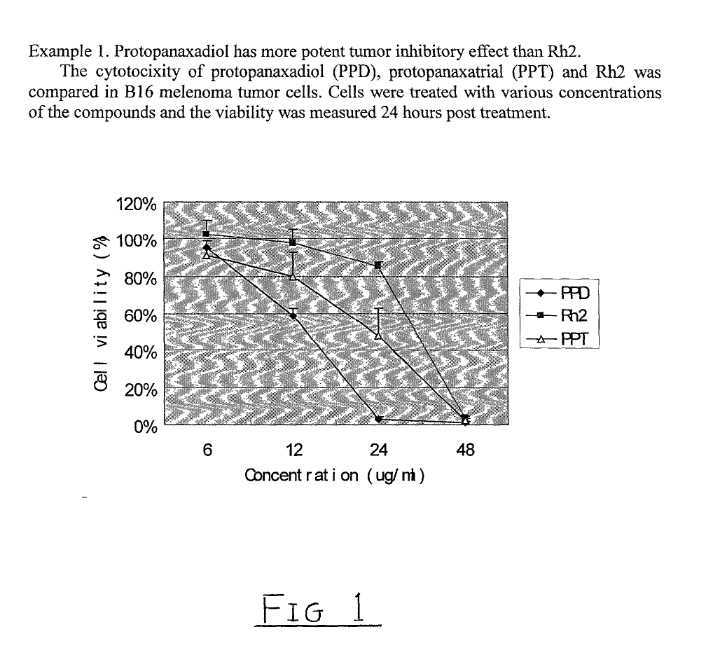

FIG. 1 shows that PPD or PPT has more potent tumor inhibitory effect than Rh2. The cytotoxicity of protopanaxadiol (PPD), or protopanaxatrial (PPT), and Rh2 was compared in B16 melanoma tumor cells. Cells were treated with various concentrations of the compounds and the viability was measured 24 hours post treatment.

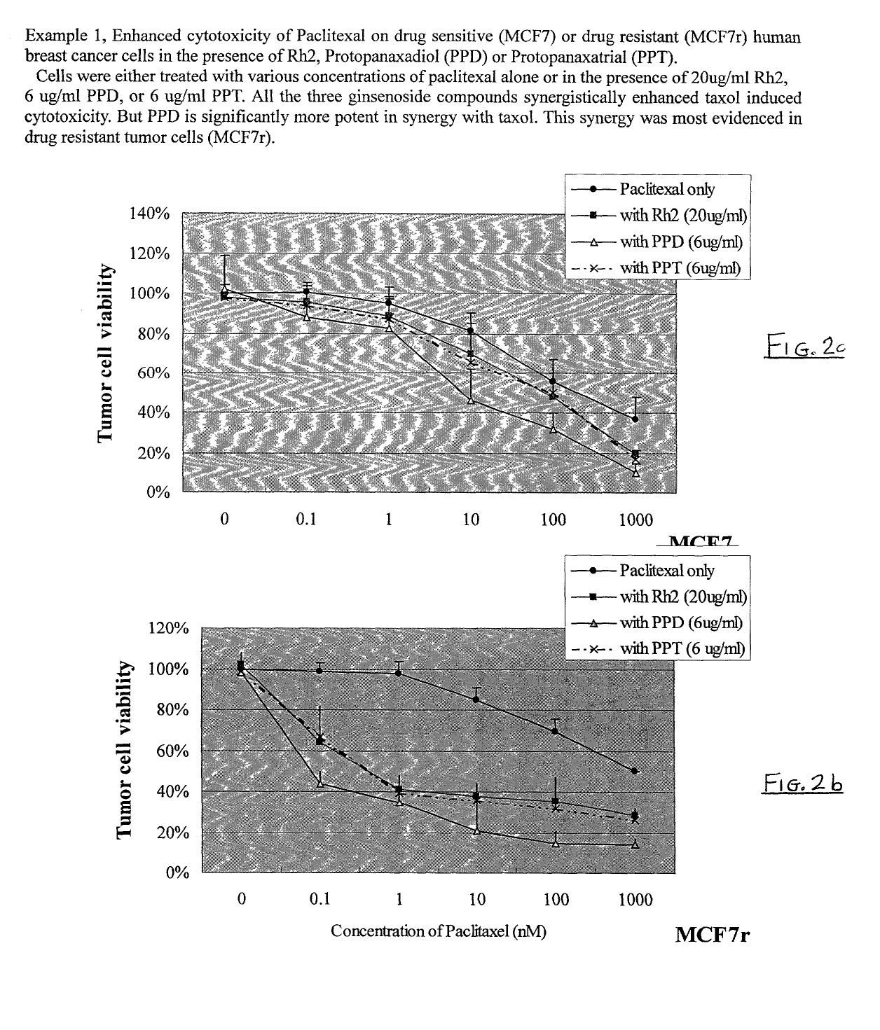

FIGS. 2a and 2b show enhanced cytotoxicity of paclitexal on drug sensitive (MCF7) or drug resistant (MCF7r) human breast cancer cells in the presence of sapogenin protopanaxadiol or sapogenin protopanaxatriol. Cells were either treated with various concentrations of paclitexal alone or in the presence of 20 μg/ml Rh2, 6 μg/ml PPT or PPD. All three of the compounds synergistically enhanced taxol induced cytotoxicity. But PPD is significantly more potent in synergy with taxol. This synergy was most prominent in drug resistant tumor cells (MDF7r).

The following table illustrates enhancement effect of Rh2, sapogenin protopanaxadiol and sapogenin protopanaxatriol on IC50 of taxol on drug sensitive (MCF7) and drug resistant (MCF7r) cell lines. It should be noted that the enhancement effect of all three compounds was much more dramatic in drug resistant cell lines than in the drug sensitive cells, especially PPD and PPT.

TABLE 1

| | | | | | Proto- | | Proto- | |
|---|---|---|---|---|---|---|---|---|
| Cell | | | Rh2 | | panaxadiol | | panaxatrial | |
| Line | Taxol only | | (20 μg/ml) | | (6 μg/ml) | | (6 μg/ml) | |
| MCF7 | 225 | nM | 61.6 | nM | 12.5 | nM | 45.8 | nM |
| | | | (3.65 | fold) | (18 | fold) | (4.9 | fold) |
| MCF7r | 929.8 | nM | 0.208 | nM | 0.0315 | nM | 0.533 | nM |
| | | | (4470 | fold) | (29517 | fold) | (1744 | fold) |

Although various embodiments of the invention are disclosed herein, many adaptations and modifications may be made within the scope of the invention in accordance with the common general knowledge of those skilled in this art. For example, the present invention comprehends all optical isomers and racemic forms of sapogenin protopanaxadiol and/or sapogenin protopanaxatriol. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”.

What is claimed is:

1. A method of inhibiting the multiplication of cancer cells comprising treating the cells with a therapeutically and synergistically effective combination of:
(a) paclitaxel and sapogenin protopanaxadiol and sapogenin protopanaxatriol;
(b) paclitaxel and sapogenin protopanaxadiol; or
(c) paclitaxel and sapogenin protopanatriol.

2. A method of inhibiting the multiplication of cancer cells comprising treating the cells with a therapeutically and synergistically effective combination of:
(a) mitoxantrone and sapogenin protopanaxadiol and sapogenin protopanaxatriol;
(b) mitoxantrone and sapogenin protopanaxadiol; or
(c) mitoxantrone and sapogenin protopanaxatriol.

3. The method of claim 1 wherein the cancer cells are multi-drug resistant.

4. The method of claim 2 wherein the cancer cells are multi-drug resistant.

5. The method of claim 3 wherein the cancer cells do not express P-glycoprotein.

6. The method of claim 4 wherein the cancer cells do not express P-glycoprotein.

7. The method of claim 1 wherein the cancer cells are selected from the group consisting of prostate cancer cells and breast cancer cells.

8. The method of claim 2 wherein the cancer cells are selected from the group consisting of prostate cancer cells and breast cancer cells.

9. A method of treating a patient having a cancer, comprising treating the patient with a therapeutically and synergistically effective combination of paclitaxel and sapogenin protopanaxadiol or paclitaxel and sapogenin protopanaxatriol, or paclitaxel and sapogenin protopanaxadiol and sapogenin protopanaxatriol.

10. A method of treating a patient having a cancer, comprising treating the patient with a therapeutically and synergistically effective combination of mitoxantrone and sapogenin protopanaxadiol or mitoxantrone and sapogenin protopanaxatriol, or mitoxantrone and sapogenin protopanaxadiol and sapogening protopanaxatriol.

11. The method of claim 9 wherein the cancer is a multi-drug resistant cancer.

12. The method of claim 10 wherein the cancer is a multi-drug resistant cancer.

13. The method of claim 11, wherein the cancer cells do not express P-glycoprotein.

14. The method of claim 12, wherein the cancer cells do not express P-glycoprotein.

15. The method of claim 9 wherein the cancer is selected from the group consisting of prostate cancer and breast cancer.

16. The method of claim 10 wherein the cancer is selected from the group consisting of prostate cancer and breast cancer.

17. The use of a therapeutically effective amount of sapogenin protopanaxadiol or sapogenin protopanaxatriol alone or together in combination with paclitaxel to synergistically inhibit the multiplication of cancer cells.

18. The use of sapogenin protopanaxadiol or sapogenin protopanaxatriol alone or together to formulate a medicament with paclitaxel for synergistically inhibiting the multiplication of cancer cells.

19. The use of a therapeutically effective amount of sapogenin protopanaxadiol or sapogenin protopanaxatriol alone or together in combination with mitoxantrone to synergistically inhibit the multiplication of cancer cells.

20. The use of sapogenin protopanaxadiol or sapogenin protopanaxatriol alone or together to formulate a medicament with mitoxantrone for synergistically inhibiting the multiplication of cancer cells.

21. The use of sapogenin protopanaxadiol or sapogenin protopanaxatriol according to claim 17 wherein the cancer cells are multi-drug resistant.

22. The use of sapogenin protopanaxadiol or sapogenin protopanaxatriol according to claim 18 wherein the cancer cells are multi-drug resistant.

23. The use of sapogenin protopanaxadiol or sapogenin protopanaxatriol according to claim 17 wherein the cancer cells do not express P-glycoprotein.

24. The use of sapogenin protopanaxadiol or sapogenin protopanaxatriol according to claim 18 wherein the cancer cells do not express P-glycoprotein.

25. The use of sapogenin protopanaxadiol or sapogenin protopanaxatriol according to claim 17 wherein the cancer cells are selected from the group consisting of prostate cancer cells and breast cancer cells.

26. The use of sapogenin protopanaxadiol or sapogenin protopanaxatriol according to claim 18 wherein the cancer cells are selected from the group consisting of prostate cancer cells and breast cancer cells.

27. A method of inhibiting the multiplication of multi-drug resistant cancer cells, wherein the cells do not express P-glycoprotein, comprising treating the cells with a therapeutically and synergistically effective combination of sapogenin protopanaxadiol or sapogenin protopanaxatriol alone or together and a chemotherapeutic.

28. The method of claim 27 wherein the chemotherapeutic is paclitaxel.

29. The method of claim 27 wherein the chemotherapeutic is mitoxantrone.

30. The method of claim 27 wherein the chemotherapeutic is cisplatin.

31. The method of claim 27 wherein the multi-drug resistant cancer cells are selected from the group consisting of prostate cancer cells and breast cancer cells.

32. The use of a therapeutically effective amount of sapogenin protopanaxadiol or sapogenin protopanaxatriol alone or together in combination with a chemotherapeutic to inhibit the multiplication of multi-drug resistant cancer cells, wherein the cells do not express P-glycoprotein, and the chemotherapeutic alone is not therapeutically effective to inhibit the multiplication of the cancer cells.

33. The use of sapogenin protopanaxadiol and sapogenin protopanaxatriol singly or together in accordance with claim 32, wherein the chemotherapeutic is paclitaxel.

34. The use of sapogenin protopanaxadiol or sapogenin protopanaxatriol singly or together in accordance with claim 32, wherein the chemotherapeutic is mitoxantrone.

35. The use of sapogenin protopanaxadiol or sapogenin protopanaxatriol singly or together in accordance with claim 32, wherein the chemotherapeutic is cisplatin.

36. The use of sapogenin protopanaxadiol and sapogenin protopanaxatriol according to claim 32 wherein the multi-drug resistant cancer cells are selected from the group consisting of prostate cancer cells and breast cancer cells.

37. A pharmaceutical composition comprising: a pharmaceutically acceptable carrier; and, therapeutically and synergistically effective amounts of sapogenin protopanaxadiol or sapogenin protopanaxatriol singly or in combination and a chemotherapeutic selected from the group consisting of paclitaxel and mitoxantrone.

38. The pharmaceutical composition of claim 37, wherein the chemotherapeutic is paclitaxel.

39. The pharmaceutical composition of claim 37, wherein the chemotherapeutic is mitoxantrone.

40. The pharmaceutical composition of claim 37, wherein the form of the composition is selected from the group consisting of an orally administrable form, an injectable form, an externally applicable form, a food form, a multi-nutritional product form and an alternative medicine form.

41. The pharmaceutical composition of claim 40, wherein the composition is the orally administrable form and is selected from the group consisting of a tablet, a powder, a suspension, an emulsion, a capsule, a granule, a troche, a pill, a liquid, a spirit, a syrup and a limonade.

42. The pharmaceutical composition of claim 40, wherein the composition is the injectable form and is selected from the group consisting of a liquid, a suspension and a solution.

43. The pharmaceutical composition of claim 40, wherein the composition is the externally applicable form and is selected from the group consisting of an ointment, a liquid, a powder, a plaster, a suppository, an aerosol, a liniment, a lotion, an enema and an emulsion.
